(19) United States
(12) Patent Application Publication
(10) Pub. No.: US 2026/0102285 A1
(43) Pub. Date: Apr. 16, 2026

(54) PASSIVE INTRAOCULAR PRESSURE CONTROL AND ASSOCIATED SYSTEMS, DEVICES, AND METHODS

(71) Applicant: Verily Life Sciences LLC, Dallas, TX (US)

(72) Inventors: Amirhossein Samakar, South San Francisco, CA (US); Allister McGuire, South San Francisco, CA (US)

(21) Appl. No.: 19/418,736

(22) Filed: Dec. 12, 2025

Related U.S. Application Data
(63) Continuation of application No. 17/709,552, filed on Mar. 31, 2022.
(60) Provisional application No. 63/170,293, filed on Apr. 2, 2021.

(51) Int. Cl. A61F 9/007; A61N 1/36; A61N 1/372; A61N 1/375

(52) U.S. Cl. CPC A61F 9/00781; A61N 1/36046; A61N 1/37223; A61N 1/375

(57) ABSTRACT

Devices, systems, and methods for passively regulating or controlling Intraocular Pressure (IOP) are provided. A device may be configured to be implanted or attached to a patient’s eye to provide regulated drainage of aqueous humor (AH) out of the eye to an exterior of the eye. The device may include a housing comprising an inlet and an outlet, and a pressure relief valve coupled to the housing. The pressure relief valve may be configured to open to allow passage of the aqueous humor from an ingress of the pressure relief valve to an egress of the pressure relief valve in response to a pressure of the aqueous humor on the ingress exceeding a threshold. The device may include a filter configured to allow passage of fluid from the egress through the outlet along a fluid path extending from the inlet to the outlet.

### CROSS-REFERENCE TO RELATED APPLICATIONS

The present Application is a continuation of U.S. Patent Application No. 17/709,552, filed March 31, 2022, which claims the benefit of and priority to U.S. Provisional Patent Application No. 63/170,293, filed April 2, 2021, all of which are hereby incorporated by reference herein in their entirety.

### TECHNICAL FIELD

The present disclosure relates generally to controlling Intraocular Pressure (IOP) using passive pressure relief devices, and associated systems and methods.

### BACKGROUND

Intraocular pressure (IOP) quantifies the pressure of fluid inside the eye. Many individuals suffer from disorders, such as glaucoma, that cause chronic heightened IOP. In addition to pain and discomfort, heightened IOP can, if not adequately treated, cause damage to the optical nerve of the eye, leading to loss of vision. Presently, treatment of glaucoma mainly involves periodically administering pharmaceutical agents to the eye to decrease IOP. These drugs can be delivered, for example, by injection or eye drops. However, effective treatment of glaucoma may involve adherence to dosage schedules and a knowledge of the patient’s IOP. The IOP for a given patient can vary significantly based on time of day, exercise, recency of medication use, and other factors. Thus, conventional approaches to treating IOP may yield varying results, and may fail to consistently maintain IOP at a stable level.

Drainage devices, such as shunts, have been developed to allow aqueous humor (AH) – the fluid inside the eye – to be drained from the interior cavities of the eye. Such devices are surgically implanted and include a small cannula or tube which leads from the interior of the eye to a tissue-bound capsule, referred to as a bleb, closer to the surface of the eye. The structure of the bleb naturally provides resistance to AH flowing out of the eye, which provides some regulation of the flow to keep IOP pressures from decreasing too quickly. Over time, the bleb can fibrose, increasing the resistance to, or even blocking the egress of, AH. Thus, the effectiveness of the drainage device can decrease. Additional surgeries may be required to correct the blockage, or form a new bleb.

### SUMMARY

The present disclosure advantageously describes devices, systems, and methods for regulating or controlling Intraocular Pressure (IOP). According to some aspects, a device is presented that is configured to be implanted or attached to a patient’s eye to provide regulated drainage of aqueous humor (AH) out of the eye into the tear film of the eye, which is an exterior surface of the eye. In some embodiments, the device includes a housing having an inlet and an outlet, a pressure relief valve configured to allow for passive regulation of IOP, and a filter positioned between an egress of the pressure relief valve and the outlet to provide a barrier for bacteria, viruses, or other bodies or substances from back flowing through the device into the eye.

According to one aspect of the present disclosure, a therapeutic device configured to be worn on an eye of a patient includes: a housing comprising an inlet and an outlet, the inlet configured for receiving aqueous humor from the eye; a pressure relief valve coupled to the housing, where the pressure relief valve is configured to open to allow passage of the aqueous humor from an ingress of the pressure relief valve to an egress of the pressure relief valve in response to a pressure of the aqueous humor on the ingress of the pressure relief valve exceeding a threshold; and a filter coupled to the housing and positioned between the egress of the pressure relief valve and the outlet of the housing, such that the filter is configured to allow passage of fluid from the egress of the pressure relief valve through the outlet of the housing along a fluid path extending from the inlet of the housing to the outlet.

In some embodiments, the therapeutic device further includes: one or more electrodes coupled to the housing adjacent to the filter and in fluid communication with the filter; and electronic circuitry coupled to the housing and configured to apply a voltage to the one or more electrodes to remove a filter cake from a surface of the filter. In some embodiments, the one or more electrodes includes a first electrode and a second electrode, and the filter is positioned between the first electrode and the second electrode. In some embodiments, the first electrode, the second electrode, and the filter comprise a same footprint. In some embodiments, the therapeutic device further includes: an antenna coupled to the housing and in electrical communication with the electronic circuitry, where the antenna is configured to wirelessly receive electromagnetic energy, and provide an electrical current to the electronic circuitry.

In some embodiments, the therapeutic device further includes:an inlet filter coupled to the housing and positioned between the ingress of the pressure relief valve and the inlet of the housing, where a pore size of the inlet filter is larger than a pore size of the filter. In some embodiments, the pore size of the inlet filter is less than 10 μm. In some embodiments, the pressure relief valve includes a spring-biased membrane configured to open to allow passage of the aqueous humor in response to the pressure at the ingress of the pressure relief valve exceeding an opening pressure associated with the spring-biased membrane.

According to another embodiment of the present disclosure, a device configured to be worn on an eye of a patient includes: a housing comprising a first inlet and an outlet, the first inlet configured for receiving aqueous humor from an interior the eye, and the outlet configured for delivering the aqueous humor to a tear film of the eye. The device further includes a passive valve positioned within the housing and configured to open to allow passage of the aqueous humor from an ingress of the passive valve to an egress of the passive valve in response to a pressure of the aqueous humor at the ingress of the passive valve exceeding a first threshold. The device further includes a first filter positioned within the housing between the egress of the passive valve and the outlet, and one or more electrodes positioned within the housing adjacent to the first filter and in fluid communication with the first filter. The device further includes electronic circuitry coupled to the housing and configured to apply a voltage to the one or more electrodes. The one or more electrodes, in response to the applied voltage, are configured to remove debris from a surface of the first filter, and the passive valve is positioned in a fluid path between the first filter and the outlet.

In some embodiments, the electronic circuitry includes a pressure sensor configured to monitor a pressure of the aqueous humor. In some embodiments, the pressure sensor is configured to monitor the pressure at the first inlet. In some embodiments, the device further includes a second filter positioned within the housing between the ingress of the passive valve and the first inlet of the housing, where the first filter comprises a first pore size, and where the second filter comprises a second pore size larger than the first pore size.

In some embodiments, the device further includes a filter electrode positioned within the housing adjacent to the second filter and in fluid communication with the second filter, where the electronic circuitry is configured to apply a further voltage to the filter electrode, and where the filter electrode, in response to the further voltage, is configured to remove debris from a surface of the second filter. In some embodiments, the electronic circuitry further includes a processor in communication with the pressure sensor, where the processor is configured to apply the further voltage to the filter electrode in response to determining that the pressure of the aqueous humor at the first inlet of the housing exceeds a second threshold, where the second threshold is greater than the first threshold.

In some embodiments, the device further includes a pump configured to receive electrical power from the electronic circuitry, where the housing further comprises a second inlet configured for receiving the aqueous humor from the interior the eye, and where the pump is configured to pump the aqueous humor through the second inlet toward the second filter. In some embodiments, the pump is configured to direct the debris removed from the second filter through the first inlet to return to the eye. In some embodiments, the one or more electrodes comprises a first electrode and a second electrode, where the first filter is positioned between the first electrode and the second electrode, and where the electronic circuitry is configured to apply the voltage between the first electrode and the second electrode.

According to another embodiment of the present disclosure, a device configured to be worn on an eye of a patient includes a housing comprising an inlet and an outlet, the inlet configured for receiving aqueous humor from an interior the eye, and the outlet configured for delivering the aqueous humor to an exterior of the eye. The device further includes a filter positioned within the housing and configured to separate substances in the aqueous humor from the aqueous humor, a first electrode positioned within the housing adjacent to the filter, and a second electrode positioned within the housing adjacent to the filter, where the filter is positioned between the first electrode and the second electrode. The device further includes a pressure sensor configured to monitor a pressure of the aqueous humor within the housing, and electronic circuitry positioned within the housing and configured to induce a voltage between the first electrode and the second electrode to remove debris from a surface of the filter. The device further includes an antenna positioned within the housing and configured to receive electromagnetic energy to provide an electrical current to the electronic circuitry.

In some embodiments, the electronic circuitry comprises a processor configured to: cause the pressure sensor to monitor the pressure in response to receiving the electrical current from the antenna; determine whether the pressure exceeds a threshold; and apply the voltage between the first electrode and the second electrode in response to determining that the pressure exceeds the threshold. In some embodiments, the pressure sensor is configured to monitor the pressure within the inlet of the housing.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a perspective view of an intraocular pressure (IOP) regulation device, according to aspects of the present disclosure.

FIG. 2 is a diagrammatic view of an IOP regulation device, according to some aspects of the present disclosure.

FIG. 3 is a diagrammatic view of an IOP regulation device, according to aspects of the present disclosure.

FIG. 4 is a cross-sectional, exploded view of an IOP regulation device, according to an embodiment of the present disclosure.

FIG. 5 is an exploded view of a passive pressure relief valve, according to an embodiment of the present disclosure.

FIG. 6 is a diagram of electronic circuitry of a passive IOP regulation device, according to aspects of the present disclosure.

FIG. 7 is a diagram of electronic circuitry of a passive IOP regulation device, according to aspects of the present disclosure.

FIG. 8A is a diagrammatic view of an IOP regulation device during normal operation, according to some aspects of the present disclosure.

FIG. 8B is a diagrammatic view of an IOP regulation device during a cleaning operation, according to some aspects of the present disclosure.

FIG. 9 is a flow diagram of a method for cleaning an IOP regulation device, according to some aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

Disclosed herein are devices for implanting in or otherwise attaching to an eye. The devices, systems, and methods described herein may be used to reduce, regulate, adjust, or otherwise control intraocular pressure (IOP) in the treatment of ophthalmic conditions such as glaucoma. In some aspects, the devices described herein may include pressure relief valves configured to passively regulate IOP. The valves may be configured to open to allow egress of aqueous humor (AH) from inside the eye when the IOP equals or exceeds an opening pressure of the pressure relief valve. The device is configured to drain the egressed AH out of the eye to the tear film. Further, the device may include a filter, or a combination of filters, positioned at or adjacent to an egress and/or an ingress of the valve, such that the filter prevents foreign materials (e.g., bacteria, viruses) from traveling backward into the eye. The device may further include one or more electrodes positioned adjacent to the filter or filters. The electrodes can be activated to remove clogs or materials stuck to a surface of a filter using electrophoresis, electrolysis, or any other suitable approach to break down and disperse the clogging particles away from the filter. The device may be passively controlled using a wireless remote control device brought within an operating distance of the eye.

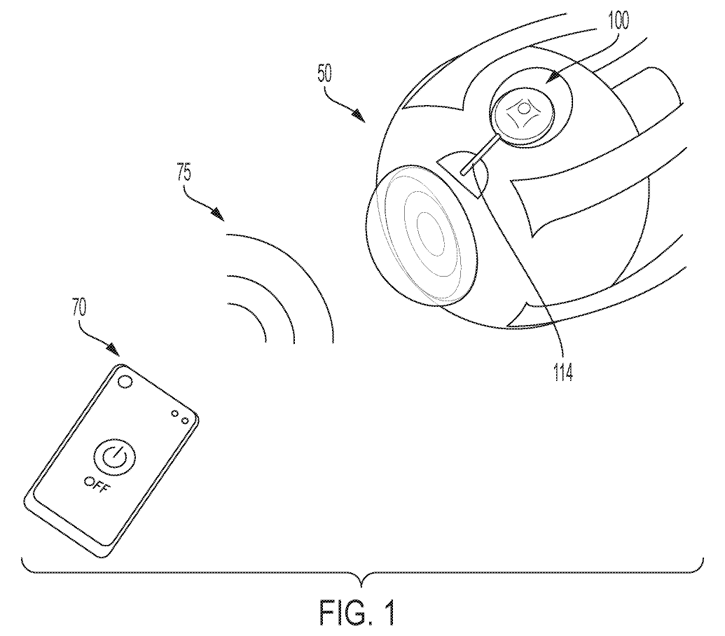

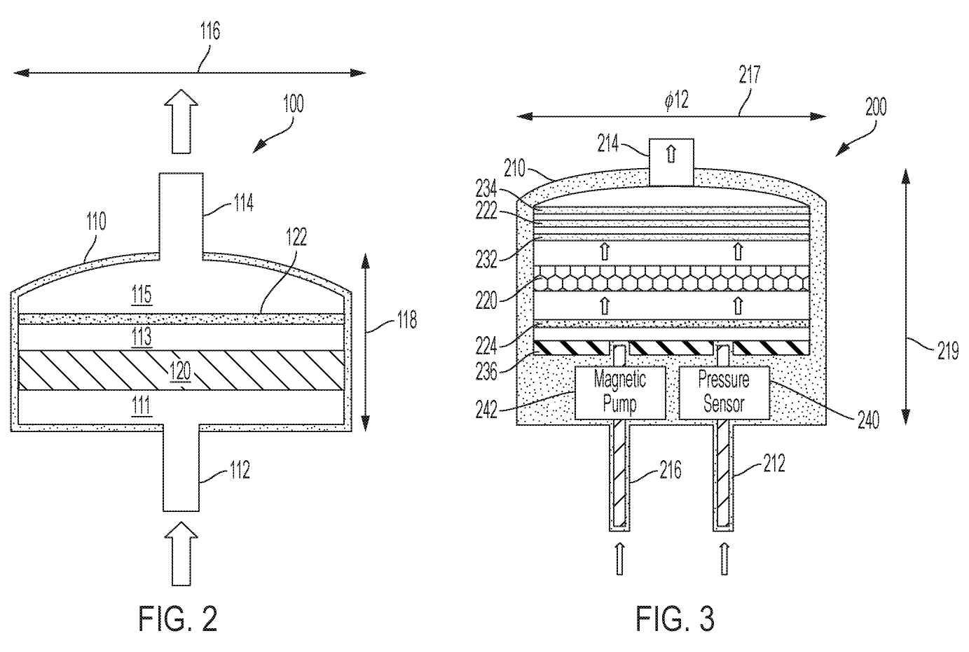

FIG. 1 is a perspective view of an IOP regulation device 100, which is implanted or attached to a human eye 50. The device 100 includes a relatively flat, smooth outer profile to fit within the periocular space around the eye 50. The device 100 includes an inlet (see, e.g ., 112, FIG. 2), and an outlet 114. For example, the inlet in FIG. 1 may be located underneath the body of the device 100 and inserted at least partially into the eye 50. The device 100 is configured to allow egress of AH from the eye 50 in response to the IOP of the eye 50 meeting or exceeding a threshold. As will be further explained below, the device may include a pressure relief valve and a filter positioned within, and encapsulated by, a housing. The threshold may include or otherwise correspond to an opening pressure of a pressure relief valve. The device 100 may be implanted by a physician and positioned such that the majority or entirety of the device is not visible to others. The outlet 114 may be positioned to allow drainage of the AH into the tear film. Accordingly, the AH may drain to an exterior surface of the eye 50, rather than a bleb, for example.

The device 100 may be controlled by a wireless remote control device 70. The wireless remote device 70 provides power in the form of electromagnetic waves or energy 75. The electromagnetic energy 75 may pass through an antenna of the device 100 as further described below. The antenna may harvest the electromagnetic energy 75 and convert the electromagnetic energy 75 into an electrical current or voltage. The electrical current or voltage is provided to the electronic components of the device 100, including one or more electrodes for filter cleaning, a pressure sensor, a pump, and/or any other suitable components. In some aspects, the wireless remote control device 70 may also provide instructions to the device 100, and/or receive data from the electronic components of the device 100. The wireless remote device 70 may be configured such that the electromagnetic energy 75 remains below safety thresholds established by government agencies such as the U.S. Food and Drug Administration (FDA), for example.

In some aspects, the wireless remote control device 70 may be configured with smart activation features. The wireless remote control device 70 may include a smartphone, or may provide for wireless connectivity with the smartphone (e.g., Bluetooth®) using a smartphone app. The remote control device 70 may include a memory storing a variety of stimulation waveforms for electromagnetic pulsing and algorithms. The wireless control device 70 may include various tracking features, such as an accelerometer to track the remote control device’s 70 motion, and/or a wireless connection with a cellphone. The wireless remote control device 70 may track cleaning time(s) and duration, and send reminders. In some embodiments, the device 100 may include an electrical power storage, such as a battery or a capacitor, configured to provide power to one or more components of the device 100 when the remote control device 70 is not providing power to the device 100.

The components of the device 100 may be contained within, and encapsulated by, a housing. The housing may be flexible, or rigid. For example, the housing may include a flexible polymer encasing the components of the device, providing for electrical insulation, and isolation from the fluids of the eye and the periocular space. The housing may be deformed (e.g., heat shrunk, stamped, etc.) around the components to provide for a smaller overall profile. The housing may be biocompatible and smooth to reduce irritation and/or tissue damage to the eye 50, which is almost constantly in motion.

FIG. 2 is a diagrammatic view of an IOP regulation device 100, according to embodiments of the present disclosure. In the illustrated embodiment, the device 100 includes a housing 110 having an inlet 112 and an outlet 114, where the housing 110 encases or houses a pressure relief valve 120 and a filter 122. The inlet 112 is configured to insert at least partially into an interior of a patient’s eye (e.g., 50, FIG. 1). The inlet 112 may be surgically inserted or implanted by a physician such that the inlet 112 does not interfere with the patient’s vision, and is otherwise invisible to others. The inlet 112 and outlet 114 may include flexible tubings or cannulas extending from a main body of the housing 110. In some aspects, the inlet 112 and outlet 114 are distinct components which are attached to the main body of the housing 110. In other embodiments, the inlet 112, and/or the outlet 114 are integral with the main body of the housing 110, such that the housing 110, including the inlet 112 and the outlet 114, comprises an integral or monolithic structure. The housing 110 has a diameter or width 116, and an effective height 118. The effective height 118 may refer to a height or thickness of the main body of the housing. For instance, it will be understood that the inlet 112 and/or the outlet 114 are illustrated in diagram form in FIG. 2, and may not add to the height or thickness of the device 100 once implanted on the eye. For example, the inlet 112 and/or the outlet 114 may extend from one or more lateral sides of the device, as shown in FIG. 1. The diameter 116 and height 118 may be suitable for implantation on the eye, and can be worn permanently or semi-permanently in the periocular space between the eye and periocular tissue (e.g., eyelid). In some embodiments, the diameter or width 116 may range from 10 mm to 1 mm, including values such as 5 mm, 4 mm, 3 mm, or any other suitable value, both greater and smaller. The height 118 may range from approximately 5 mm to 0.5 mm, including values such as 2 mm, 1 mm, or any other suitable value, both greater and smaller.

The pressure relief valve 120 may include a passive, mechanical valve configured to open at least partially in response to a fluid pressure at an ingress area 111 of the valve 120 matching or exceeding a threshold, which may be an opening pressure of the valve 120. For example, the valve 120 may include a resilient, biased membrane having a default closed position in which the membrane or another component coupled to the membrane forms a seal with a valve seat, for example. The valve seat may be a surface configured to engage or contact a surface of the membrane or other object under spring force provided by the membrane. In such embodiments, the valve 120 opens when the fluid pressure of the AH at the ingress 111 overcomes the spring force of the membrane. Accordingly, in some embodiments, the valve 120 is configured to operate without electrical or magnetic power. In other embodiments, the valve 120 may be operated electronically or magnetically (e.g., by wireless remote control 70).

Fluid (e.g., AH) that escapes the valve 120 travels to an egress area 113, and through a filter 122. In the illustrated embodiment, the filter 122 may be described as distal of the valve 120 along a fluid flow path extending from the inlet 112 to the outlet 114. Although the valve 120 and the filter 122 are shown in a stacked configuration and the fluid flow path is shown as linear, it will be understood that the fluid flow path may not necessarily be linear. For example, in some embodiments, the valve 120 and filter 122 may be arranged in a non-stacked configuration such that at least a part of the fluid flow path is directed laterally along a surface of the valve 120 and/or filter 122. For example, the valve 120 and the filter 122 may be positioned in a staggered configuration where the outlet 114 is offset from the inlet 112.

The filter 122 may have a pore size ranging from 1 um to 0.01 um, for example. In an exemplary embodiment, the filter 122 has a pore size of 0.1 um. In such embodiments, the filter 122 may be configured to block the passage of bacteria through the filter 122. This may be particularly advantageous in the direction of reverse flow, from the outlet 114 to the inlet 112. In some instances, bacteria, viruses, or other undesirable foreign material may travel through the outlet 114 and into an egress area 115 distal of the filter 122. The filter 122 may provide a barrier for bacteria and/or viruses from entering the eye from the exterior of the eye (e.g., the tear film). The pore size of the filter 122 may be sufficiently small to block even small viruses from entering the interior of the eye (e.g., 0.02 um). Accordingly, whereas filters are typically used to separate undesirable materials in a fluid flowing along a flow direction or path, the filter 122 advantageously provides a bacterial and/or viral barrier such that viruses and bacteria are prevented from reaching even the valve 120.

As explained below, additional features may be included into the device 100, such as cleaning components configured to clean filtered substances off a surface of the filter 122 to maintain the operability of the filter and reduce or eliminate clogs. Further, additional filters may be included at different locations of the device 100, such as the ingress portion 111 to filter out smaller viruses, proteins, or other materials that may impede or degrade the performance of the valve 120 and/or the filter 122. The cleaning components may include one or more electrodes configured to induce a voltage across a filter, or between the filter and an electrode, to clean the electrode using electronic cleaning. In some aspects, an electronic cleaning process may include pulsing one or more electrodes using a frequency profile or waveform to remove and disintegrate a layer of foreign material, referred to as a filter cake, from a surface of the filter. In other aspects, the electronic cleaning process may include inducing a direct current having a constant voltage, or substantially constant voltage, between the electrodes and/or an electrode and the filter 122. Examples of electronic cleaning include electrophoretic cleaning and electrolytic cleaning.

FIG. 3 is a diagrammatic view of a passive pressure regulation device 200, according to embodiments of the present disclosure. The device 200 may include components similar or identical to the components of the device 100 shown in FIG. 2. For example, the device 200 shown in FIG. 3 includes a housing 210 having a first inlet 212 and an outlet 214, a pressure relief valve 220, and a filter 222. The pressure relief valve 220 and/or the filter 222 may be similar or identical to the valve 120 and filter 122 shown in FIG. 2, in some aspects. The device 200 shown in FIG. 3 further includes a filter 224 configured to filter materials from incoming AH before flowing to the ingress of the valve 220. The filter 224 may also provide an additional barrier from bacteria and/or viruses from flowing backward through the device 200 into the eye.

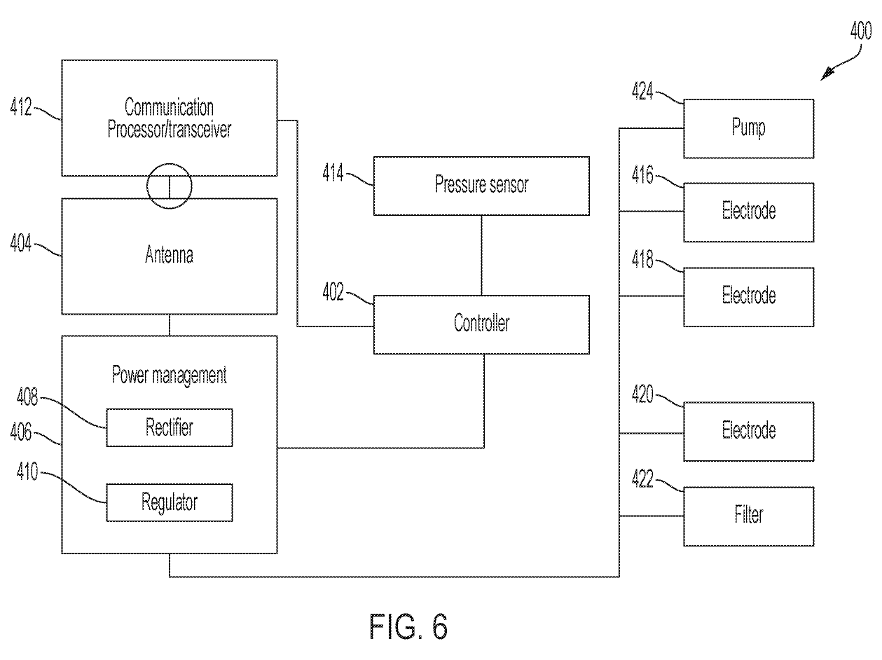

The device 200 also includes electronic cleaning components configured to facilitate the cleaning and maintenance of the filters 222 and 224. The electronic cleaning components include a first electrode 232, a second electrode 234, and a third electrode 236. The electrodes 232, 234, 236 may be controlled by electronic circuitry (see FIG. 6) on the device 200, and are configured to remove, dislodge, or otherwise clean materials that are stuck or attached to the surfaces of the filters 222, 224.

The filter 222 may comprise a pore size smaller than the pore size of the filter 224, in some embodiments. For example, the filter 222 may have a pore size of ranging from 1 um to 0.01 um, including values such as 0.05 um, 0.03 um, or 0.02 um. In an exemplary embodiment, the filter 224 has a pore size of approximately 0.1 um, and the filter 224 has a pore size of approximately 0.02 um. In this regard, the filter 222 may be configured to prevent even the smallest viruses from passing into the inlets 212, 216 of the device 200. In some aspects, the filter 224 may be configured to remove cell debris and gelatinous materials from the AH flowing into the device 200. In that regard, without the filter 224, substances in the AH could potentially cause issues with the function of the valve 220, and could potentially degrade the performance of the valve 220 over time. The filter 224 thus prevents these materials from entering the valve 220. In other embodiments, the filter 224 may have a pore size smaller than the pore size of the filter 222.

The electrodes 232, 234, 236 can be activated to remove the filtered substances from the surfaces of the respective filters 222, 224. For example, the first electrode 232 and the second electrode 234 may be activated by electronic components of the device 200 to clean the filter 222 electronically. In some aspects, the electronic components may power the electrodes 232, 234 in a pulsed pattern, with the electrodes 232, 234 being pulsed with opposite polarities. In other aspects, the electronic components of the device 200 may apply direct current (DC) and/or constant voltage to the electrodes 232, 234, 236 to clean the filters 222, 224 (e.g., using electrolysis). In some embodiments, one of the electrodes 232, 234, is a ground or neutral electrode, and the other electrode is activated or pulsed. The filters 222, 224 may include ceramic filters, polymeric filters, and/or metalized filters. For example, the filter 222 may include a ceramic filter, such as an anodic aluminum oxide filter. The filter 224 may include a metalized filter stack including a metallic cathode and a membrane. In some embodiments, the filter 224 includes multiple filter layers, including a filter layer having a relatively larger pore size (e.g., 1 um) configured to reduce clogging from larger particulates and to increase overall surface area for protein adhesion. In some embodiments, the filter 224 and/or the filter 222 includes a Whatman® Anodisc filter. In some embodiments, the filter 222 and/or the filter 224 includes a Pall® Supor® membrane disc filter, which may have a pore size between 0.1 μm and 0.8 μm. In some embodiments, the filter 222 and/or the filter 224 includes a STERLITECH polyethersulfone (PES) membrane filter, which may have a pore size between 0.02 μm and 8 μm.

A configuration in which the filters 222, 224 are positioned at different sides of the valve 220, and in which the filter 222 has a smaller pore size than the filter 224, may be advantageous in various aspects. For example, because dislodged filter cake particles can be returned to the eye, the filter 224 prevents thicker protein material (e.g., albumin) from clogging the mesh electrodes 232, 234 or interfering with the mechanics of the valve 220. Further, the filter 222 prevents even the smallest viruses from passing beyond the filter 222 into the chambers of the device 200, or into the eye.

In the illustrated embodiment, the filter 224 may be configured as an electrode of an electrode pair that includes the third electrode 236. For example, the electronic circuitry of the device 200 may be configured to activate the electrode 236 and the filter 224 with opposite polarities to clean a surface of the filter 224. The electrodes 232, 234, 236 may have a mesh structure, or other structure that includes one or more openings for fluid flow, to reduce impedance on the flow of AH through the device 200. For example, one or more of the electrodes 232, 234, 236 may include a titanium mesh. In some embodiments, the electrode 236 includes a flat electrode.

In some embodiments, the filter 224 is not configured to function as an electrode (e.g., is not metallized), and the device 200 includes an additional electrode between the filter 224 and the valve 220. Accordingly, the filter 224 may be “sandwiched” or positioned between two electrodes configured to electronically clean the filter 224, similar to the filter 222. In other embodiments, the filter 222 may be configured to function as an electrode (e.g., is metallized), and the electronic components of the device 200 may induce a voltage between the filter 222 and an electrode, such as one of the electrodes 232 or 234. Other combinations are also contemplated, such as an embodiment in which the filter 222 includes an electrode in a filter/electrode combination, and the filter 224 does not include an electrode, and is positioned between two electrodes.

The device 200 further includes a pressure sensor 240 and a pump 242, which may advantageously improve the cleaning processes. The pressure sensor 240 is configured to monitor fluid pressure within the inlet 212. In other embodiments, the pressure sensor 240 may be configured to monitor fluid pressure within other spaces of the device 200, such as a chamber adjacent to an ingress or an egress of the valve 220. Pressure increases in the device 200 that are higher than the opening pressure of the valve 220, for example, may indicate that one or both of the filters 222, 224 is clogged or covered by debris from the AH. A microcontroller may be configured to continuously compare the pressure measurements obtained by the pressure sensor 240 to a threshold. In some aspects, the threshold may be greater than the opening pressure of the valve 220, indicating impedance in the device 200 other than the valve 220. The pressure sensor 240 may include a diaphragm pressure sensor, a piezoelectric pressure sensor, a micro electromechanical pressure sensor, or any other suitable type of pressure sensor. For example, in some embodiments, the pressure sensor 240 includes an SMI 95-G sensor..

The pump 242 may also assist in the cleaning processes. The pump 242 is in communication with a second inlet 216, and is configured to pump AH into the chamber of the device 200 through the second inlet 216. The pump 242 may include a magnetic coil and an armature configured to respond to magnetic fields induced by the coil. For example, the pump 242 may include a magnetic micropump as described in U.S. Provisional Application No. 63/270,398, filed October 21, 2021, the entirety of which is incorporated by reference herein. In some embodiments, when the third electrode 236 and/or the filter 224 are activated to induce a pulsed voltage between the filter 224 and the electrode 236, the filter cake is broken down and removed from a surface of the filter 224. To expel the removed filter cake from the inside of the device 200, the pump 242 may be activated to move more AH through the second inlet 216 into the interior chamber of the device 200, proximal of the filter 224, and the increased pressure may cause the removed filter cake to be expelled back into the eye through the first inlet 212. In some aspects, the pump 242 may be configured to increase the fluid pressure proximal of the filter 224 to a value higher than the IOP to counter the fluid pressure coming from the eye and force the removed filter cake material back into the eye. In other embodiments, instead of pumping the AH and filter cake particles into the housing 210, the pump 242 may be configured to pump the AH and filter cake particles directly back into the eye through the second inlet 216.

As similarly described above, the first inlet 212 is configured for insertion at least partially into an interior of a patient’s eye (e.g., 50, FIG. 1). The second inlet 216 is also configured for insertion at least partially into an interior of the patient’s eye. The first inlet 212 and the second inlet 216 may be surgically inserted or implanted by a physician such that the inlets 212, 216 do not interfere with the patient’s vision, and/or are otherwise invisible to others. The inlets 212, 216 and outlet 214 may include flexible tubings or cannulas extending from a main housing body 210. In some aspects, the inlets 212, 216 and outlet 214 are distinct components which are attached to the main housing body. In other embodiments, the inlets 212, 216 and/or the outlet 214 are integral with the main housing body, such that the housing 210 comprises an integral or monolithic structure. The housing 210 includes a diameter or width 217, and an effective height 219. The effective height 219 may refer to a height or thickness of the main body of the housing 210. It will be understood that the inlets 212, 216 and outlet 214 are illustrated in diagram form. In some embodiments, the inlets 212, 216 and the outlet of the device 200 may not add to the height or thickness of the device 200 once implanted on the eye. For example, the inlets 212, 216 and outlet 214 may extend from one or more lateral sides of the device, as shown in FIG. 1. The diameter 217 and height 219 may be suitable for implantation on the eye, and can be worn permanently or semi-permanently in the periocular space between the eye and periocular tissue (e.g., eyelid). In some embodiments, the diameter or width 217 may range from 10 mm to 1 mm, including values such as 5 mm, 4 mm, 3 mm, or any other suitable value, both greater and smaller. The height 219 may range from approximately 5 mm to 0.5 mm, including values such as 2 mm, 1 mm, or any other suitable value, both greater and smaller.

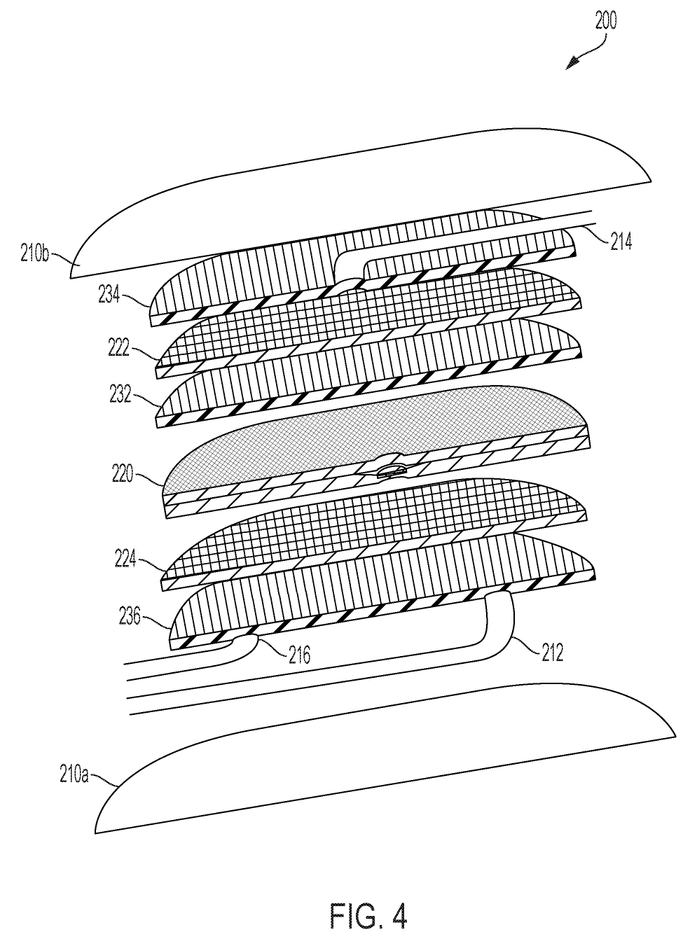

FIG. 4 is a cross-sectional, exploded view of the device 200, according to an embodiment of the present disclosure. The components of the device 200 are shown in a stacked configuration such that the footprints of each component (e.g., filters 222, 224, electrodes 232, 234, 236) are aligned along an axis extending from a first housing portion 210a to the second housing portion 210b. The inlets 212, 216 and outlet 214 extend from the device in opposing lateral directions. In some embodiments, the inlets 212, 216 may extend downward, or in an oblique direction, such that they are configured to extend through the eye tissue into the eye underneath the first housing portion 210a.

In the illustrated embodiment, the filters 222, 224, and the electrodes 232, 234, 236 comprise the same outer profile or shape, or substantially the same shape. Accordingly, the direction of fluid flow is relatively direct from the inlets 212, 216 to the outlet 214. The filters 222, 224 and the electrodes 232, 234, 236 include substantially circular/cylindrical shapes in the illustrated embodiment. In other embodiments, the filters 222, 224 and the electrodes 232, 234, 236 include elliptical, rectangular, square, triangular, or any other suitable shape or footprint. The filters 222, 224 and the electrodes 232, 234, 236 are positioned between the bottom housing portion 210a and the top housing portion 210b. The housing portions 210a, 210b may include flexible sheets or plates of material configured to be bonded together to form a hermetically sealed enclosure.

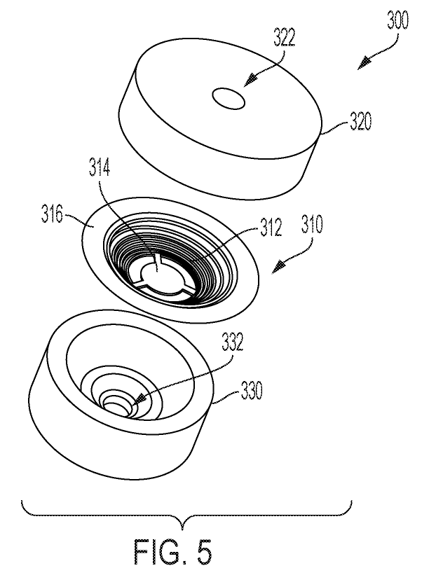

FIG. 5 is an exploded view of a passive pressure relief valve 300, according to an embodiment of the present disclosure. The valves 120 and/or 220, may include the valve 300 shown in FIG. 5, for example. The valve 300 includes a resilient spring diaphragm 310, a first body 320, and a second body 330, with the spring diaphragm 310 positioned between the first body 320 and the second body 330. The spring diaphragm 310 includes a spiral-cut spring portion 312, a sealing membrane portion 314, and a flange 316. The flange 316 may be configured to provide for a seal with the first body 320 and the second body 330. The spring diaphragm 310 includes an integral or monolithic structure. In some embodiments, the spring diaphragm 310 includes a metallic sheet (e.g., titanium) cut by laser, etching, or other suitable cutting method, to form the features shown in FIG. 5.

In some embodiments, the second body 330 includes a valve seat configured to form a valve seal with the membrane 314 of the spring diaphragm 310. The spring diaphragm 310 opens and lifts from the valve seat to allow passage of a volume of AH through the opening 332 of the second body 330 and the open spaces of the spring diaphragm 310 and out an opening 322 of the first body. The first body 320 and the second body 330 may include monolithic components formed by grinding, milling, casting, etc. In some embodiments, a plurality of valves 300 are included into a drainage device, such as the devices 100, 200 to increase fluid flow or throughput through the device.

When assembled, the valve 300 forms a cylindrical body configured to be positioned within a housing, such as the housing 210 shown in FIGS. 3 and 4, and in combination with one or more filters, and/or one or more electrodes. In some embodiments, a portion of the housing seals around the edges of the valve 300 such that fluid may only travel through the opening 332 and out the opening 322. The first body 320 and second body 330 may comprise biocompatible materials, and may include polymers and/or metallic materials. For example, the first and second bodies 320, 330 may include titanium, polyether ether ketone (PEEK), stainless steel, nickel-titanium alloy, or any other suitable material. The valve 300 may be sized and shaped to have a profile suitable to be worn on the eye within the periocular space. In some embodiments, the valve 300 includes a width or outer diameter of 1 cm or less, including values such as 5 mm, 4 mm, 3 mm, 2 mm, 1 mm. 0.5 mm, or any other suitable value, both greater and smaller. Further, the structure of the valve 300 may allow the height or thickness of the valve 300 to be relatively flat, or short, (e.g., less than 1 mm). In this regard, reducing the height or thickness of the valve 300 may improve the wearability and comfort of the device in the periocular space.

FIG. 6 is a diagram of electronic circuitry 400 of a passive IOP regulation device, according to aspects of the present disclosure. The electronic circuitry 400 may be wirelessly powered and/or controlled by a wireless remote control device, such as the wireless remote control device 70, for example. The electronic circuitry 400 may be used to clean, regulate, and/or monitor the efficiency of the passive IOP regulation device. The circuitry 400 includes a controller 402, an antenna 404, a power management circuit 406 including a rectifier and a regulator 410, a communication processor 412, a pressure sensor 414, a first electrode 416, second electrode 418, third electrode 420, a filter, or filter electrode 422, and a pump 424.

The controller 402 may include a microcontroller, which may comprise an application-specific integrated circuit (ASIC), one or more field-programmable gate arrays (FPGAs), or any other suitable processing component. The controller 402 may be configured to receive data from the pressure sensor 414 and/or the communication processor for controlling one or more aspects of the circuitry 400. Wireless power may be received by the antenna 404 and provided to the power management circuit 406. The wireless power may include electromagnetic energy, and the antenna 404 may include one or more loops of conductive traces for harnessing the electromagnetic energy and producing a current. The power management circuit 406 includes a rectifier 408 and a regulator 410. In some aspects, the power management circuit 406 further includes a MOSFET component to function with the antenna 404 to produce the electrical current. The rectifier 408 is configured to convert incoming alternating current (AC) electrical power into direct current (DC) electrical power for the components. The regulator 410 may include one or more capacitors, resistors, and/or transistors for regulating the power provided by the antenna to within operable limits of the electronic components of the circuitry 400.

The circuitry 400 further includes a pressure sensor 414 in communication with the controller 402. The pressure sensor 414 is controlled by the controller 402, and is configured to provide pressure measurements when power is provided to the circuitry 400. The controller 402 is configured to receive pressure data or pressure measurements obtained by the pressure sensor 414, and compare the pressure measurements to one or more thresholds. The threshold may be stored in a memory of the controller 402, in some embodiments. The controller may determine, based on the comparison, whether to activate the electrodes 416, 418, 420, the filter 422, and/or the pump 424 according to a cleaning program or protocol. In some embodiments, the threshold is a pressure greater than an opening pressure of a pressure relief valve, as explained above. The controller 402 may cause the electrodes 416, 418, 420, the filter 422, and/or the pump 424 to be activated via a transistor or switch connecting one or more of the electrodes 416, 418, 420, filter 422, and/or pump 424 to the power management circuit 406. In an exemplary embodiment, if the measured pressure equals or exceeds the predetermined threshold, which is greater than an opening pressure of the pressure relief valve, the controller 402 causes the power management circuit 406 to activate the electrodes. In some embodiments, the power management circuit 406 is configured to activate the electrodes and/or the filter 422 in a pulsed pattern having a frequency and amplitude profile. The parameters of the pulsed pattern may be saved in a memory of the controller 402, for example.

In some embodiments, the controller 402 is configured to activate the electrodes and/or the filter individually. In other embodiments, the controller 402 is configured to activate the electrodes and/or the filter altogether. Further, in some embodiments, the controller 402 is configured to activate the pump 424 independently of the electrodes. In other embodiments, the controller 402 is configured to activate the pump 424 together with the electrodes.

In some embodiments, the controller 402 is configured to receive and/or transmit data to/from the communication processor 412. The communication processor may be configured to receive data from the wireless remote control device 70 related to the functions of the components of the circuitry. For example, the communication processor 412 may be configured to receive operating parameters for the electrodes 416, 418, 420, filter 422, and/or the pump 424. Further, the communication processor 412 may be configured to prepare data for wireless transmission received from the controller 402. The data may include pressure measurements, operation time, power usage, and/or any other suitable data.

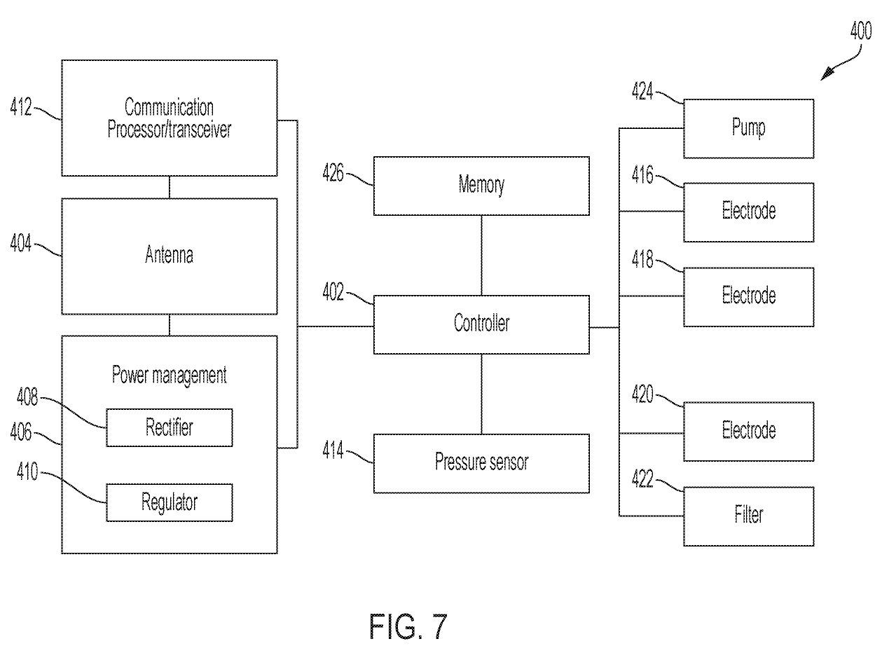

FIG. 7 is a diagram of the electronic circuitry 400 of the passive IOP regulation device, according to other embodiments of the present disclosure. In the embodiment shown in FIG. 7, the circuitry 400 is arranged with the controller 402 coupled to, and controlling, the electrodes 416, 418, 420, the filter 422, and the pump 424. The circuitry 400 further includes a memory 426 coupled to the controller 402 and configured to store operating parameters or programs for cleaning using the electrodes 416, 418, 420, the filter 422, and/or the pump 424, and/or to store pressure data obtained by the pressure sensor 414. Accordingly, instead of directly coupling the electrodes 416, 418, 420, the filter 422, and the pump 424 to the power management circuit 406 as shown in FIG. 6, the embodiment of FIG. 7 includes the controller 402 configured to direct or deliver power from the power management circuit 406 to the 416, 418, 420, the filter 422, and the pump 424.

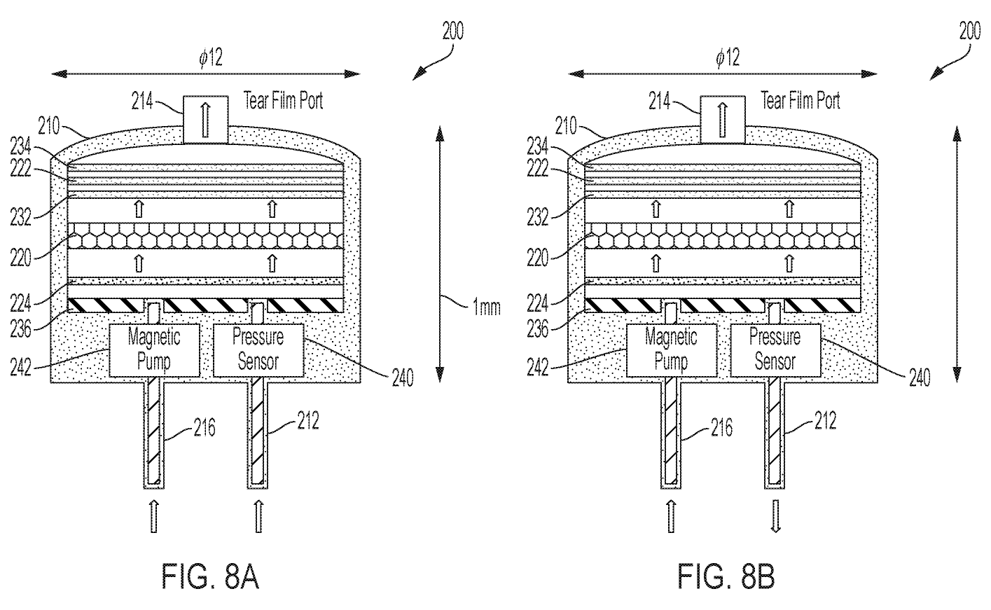

FIGS. 8A and 8B are diagrammatic views of the device 200, according to an embodiment of the present disclosure. The device 200 includes the same components shown in the embodiment of FIG. 3, including a housing 210 having a first inlet 212 and an outlet 214, a pressure relief valve 220, a first filter 222, a second filter 224, electrodes 232, 234, 236, a pressure sensor 240, and a pump 242. More specifically, FIGS. 8A and 8B illustrate the flow of AH through the device 200 in response to detecting a clog and initiating a cleaning sequence using the electrodes 232, 234, 236, and the pump 242.

FIG. 8A illustrates the device 200 during normal operation in which no clogs, or minimal clogging, of the filters 222, 224 is present. Normal operation may refer to an operating mode in which power is being provided to the device (e.g., via antenna 404 and power management circuit 406, FIG. 6), and with pressure measurements falling below a predetermined threshold. As the fluid pressure (IOP) in the inlets 212, 216 is higher than the fluid pressure at the outlet 214, the AH continues to flow inward into the housing 210 through both inlets 212, 216. In some aspects, the pressure sensor 240 may be activated or powered on to obtain pressure measurements at the inlet 212. Because the pressure measurements remain below a predefined threshold, the electrodes 232, 234, 236 are not activated. The pump 242 is also inactive. However, in some embodiments, the pump 242 may be active during normal operation in which no clogs are present.

FIG. 8B illustrates the device 200 during a cleaning sequence in which one or both of the filters 222, 224 is/are clogged. A clog at the filter 224, for example, may be the result of naturally occurring proteins in the AH binding to the porous surface of the filter 224, and occluding its opening. When the pressure sensor 240 is activated, the pressure sensor 240 obtains pressure measurements at the inlet 212, which is in fluid communication with the interior chamber of the housing 210 proximal of the filter 224. The pressure measurements may be compared to a threshold by a processor (e.g., controller 402, FIG. 6) to determine whether there is a filter clog. The threshold may be greater than an opening pressure of the pressure relief valve 220. In this regard, if the fluid pressure at the inlet 212 exceeds, and holds, at a higher value than the opening pressure of the pressure relief valve 220, it may be assumed that the filter performance of one or both of the filters 222, 224 has declined due to a clog.

In response to determining that the fluid pressure exceeds the threshold, the cleaning protocol or program is initiated. The cleaning protocol includes activating one or more of the electrodes 232, 234, 236, and/or the filter 224 in a pulsed fashion to remove a filter cake from one or both of the filters 222, 224. The filter cake may be removed from a proximal surface of the filter 224, for example, and may result in a number of filter cake particles suspended in the AH in the housing 210. To prevent the same particles from clogging the filter 224 a second time, the cleaning protocol includes activating the pump 242 to increase the fluid pressure of the AH within the housing, and particularly proximal to the filter 224. In some aspects, the pump 242 may be configured to pump AH into the housing at a rate that exceeds a flow rate of the filter 224, causing the fluid pressure to increase, and exceed the static fluid pressure associated with the IOP. As the fluid pressure increases, the AH in the housing begins flowing out of the inlet 212, and back into the eye. The AH carries with it all or a portion of the removed filter cake particles (e.g., proteins), and returns them to the eye. In other embodiments, the pump 242 may be configured to pump the AH in the opposite direction to that shown in FIG. 8B. For example, the pump 242 may be configured to pump the AH and filter cake particles directly back into the eye through the second inlet 216. This cleaning protocol can be repeated indefinitely in response to detecting that the fluid pressure at or near the inlet 212 has exceeded a predetermined threshold, thus providing for continuous filter cleaning and flushing.

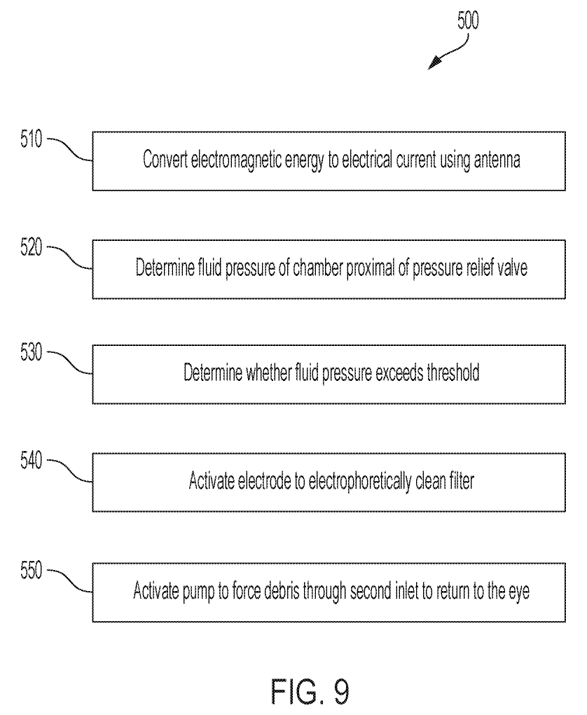

FIG. 9 is a flow diagram of a method 500 for operating an IOP regulation device, according to aspects of the present disclosure. The method 500 may be performed using one or more of the devices described above, including the devices 100, 200, the pressure relief valve 300, and/or the electronic circuitry 400. In particular, the method 500 may be performed using an IOP regulation device including an antenna, a controller or processor, a pressure sensor, one or more filters, and a pump. The method 500 may be performed to clean and/or maintain the IOP regulation device implanted into, or otherwise attached to, a patient’s eye. In some embodiments, the IOP regulation device may be passively powered by a wireless remote control device (e.g., 70, FIG. 1).

At step 510, the IOP regulation device converts electromagnetic energy to an electrical current using an antenna. For example, an antenna and a power management circuit including a rectifier may be configured to harness electromagnetic energy transmitted by a wireless communication device into an electrical current to power other components of the IOP regulation device, including a pressure sensor, controller, electrodes, and pump. Step 510 may further include regulating the current and/or voltage provided by the antenna and rectifier to within operating limits of the electronic circuitry. The electronic circuitry may include an analog and/or digital electronic configuration.

At step 520, the IOP regulation device determines a fluid pressure in a chamber of the IOP regulation device using a pressure sensor. In some embodiments, the pressure sensor is configured to obtain fluid pressure measurements of a first inlet of the IOP regulation device. In other embodiments, the pressure sensor is configured to obtain fluid pressure measurements of a chamber of the IOP regulation device that is distal of the first inlet, and proximal of a pressure relief valve. In other embodiments, the pressure sensor is configured to obtain fluid pressure measurements of a chamber of the IOP regulation device that is distal of the pressure relief valve. The IOP regulation device may be configured to power or otherwise cause the pressure sensor to obtain the pressure measurements whenever the device is powered by a wireless remote control device.

At step 530, the IOP regulation device determines whether the fluid pressure exceeds a predetermined threshold. In some embodiments, a processor or controller (e.g., controller 402, FIG. 6) is used to determine whether the fluid pressure exceeds the predetermined threshold. The threshold may be stored in a memory (e.g., memory 426, FIG. 7) coupled to the controller. The threshold may be associated with a cleaning program or protocol. In some embodiments, multiple thresholds are stored in the memory and are associated with different cleaning protocols. For example, in response to determining that the fluid pressure exceeds a first threshold, but not a second threshold, the controller may be configured to activate a first electrode, or a first subgroup of electrodes, but not a second electrode or subgroup of electrodes. In response to determining that the fluid pressure exceeds both the first threshold and the second threshold, the controller may be configured to activate all electrodes.

In some embodiments, the controller is further configured to transmit, to a communication processor, a signal indicating that the fluid pressure does not exceed the threshold. Accordingly, the signal may indicate that the filters are not clogged, or that the cleaning was successful and is complete. The communication processor may then prepare a communication signal to be transmitted to the wireless remote control device to notify the user that cleaning is either not necessary, or complete.

At step 540, in response to determining that the fluid pressure exceeds the threshold, the IOP regulation device activates one or more electrodes to electronically clean the filter. In some aspects, step 540 includes activating an electrode pair to electrophoretically clean the filter. In other aspects, step 540 includes activating an electrode pair to electrolytically clean the filter. For example, step 540 may include activating the first electrode 232 and the second electrode 234 of the device 200 to induce an electrical field across the filter 222. In other embodiments, step 540 may include activating the third electrode 236 and the filter 224. The controller may control or otherwise facilitate the activation. For example, with reference to the embodiment illustrated in FIG. 6, the controller 402 may cause the power management circuit 406 to pulse the electrodes 416, 418, 420, and/or the filter 422 based on a cleaning program or protocol saved in a memory (e.g., memory 426, FIG. 7).

At step 550, the IOP regulation device activates a pump to force debris through a second inlet to return a portion of the AH, and the particles of the filter cake removed by the cleaning process, to the eye. The pump may include a magnetic micropump including a magnetically-actuated pumping mechanism configured to direct AH from the eye into the IOP regulation device housing through the second inlet to increase the fluid pressure within the housing and cause the AH and filter cake particles to flow back into the eye through the first inlet. In some embodiments, the pump may be configured to pump in a reverse direction to pump the AH and filter cake particles directly back into the eye through the second inlet.

The IOP regulation device may be configured to perform the steps of the method 500 automatically in response to receiving power from a wireless remote control device. In some aspects, program code is stored on a memory device, where the program code includes code to cause the controller, pressure sensor, electrodes, pump, and other components to carry out the steps of the method 500. In other embodiments, the electronic circuitry may have an analog or passive configuration in which the operating parameters are only stored in the wireless remote control device, and the cleaning process is controlled based on the electromagnetic signals and waveforms transmitted by the wireless remote control device. In some aspects, the IOP regulation device is powered and the instructions for electrode cleaning are carried out as long as the wireless remote control device is providing power and/or instructions to the system within an operable range. The patient may provide wireless power to the device themselves, or it may be administered by a physician. In an exemplary embodiment, the wireless remote control device is used by the patient themselves to activate the device at home, at a time when symptoms are noticed, according to a predetermined schedule, and/or at a time otherwise appropriate or convenient to the patient.

Although the embodiments of the present disclosure are described with respect to regulating IOP (e.g., for glaucoma patients), it will be understood that the devices, systems, and methods described herein may be used to treat other ophthalmic conditions instead of or in addition to high IOP. For example, because the devices described herein can drain AH into the tear film, the devices described herein may be configured for treating dry eye conditions. Further, although the electrode cleaning processes are mostly described in the context of electrophoretic cleaning, it will be understood that the cleaning electrodes may be used for other types of cleaning processes, including electrochemical cleaning and electrolysis.

Persons skilled in the art will recognize that the devices, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A therapeutic device configured to be worn on an eye of a patient, the therapeutic device comprising:
a housing comprising an inlet and an outlet, the inlet configured for receiving aqueous humor from the eye, and the outlet configured for delivering the aqueous humor outside of the eye to an exterior surface of the eye;
a passive pressure relief valve coupled to the housing, wherein the passive pressure relief valve is configured to open from a default closed configuration to passively regulate a flow rate of the aqueous humor from an ingress of the passive pressure relief valve to an egress of the passive pressure relief valve in response to a pressure of the aqueous humor on the ingress of the passive pressure relief valve exceeding a threshold associated with the default closed configuration, wherein the passive regulation of the flow rate is configured to change in response to a change of the pressure of the aqueous humor; and
a filter coupled to the housing and positioned between the egress of the passive pressure relief valve and the outlet of the housing, such that the filter is configured to allow passage of fluid from the egress of the passive pressure relief valve through the outlet of the housing along a fluid path extending from the inlet of the housing to the outlet.

2. The therapeutic device of claim 1, further comprising:
one or more electrodes coupled to the housing adjacent to the filter and in fluid communication with the filter; and
electronic circuitry coupled to the housing and configured to apply a voltage to the one or more electrodes to remove a filter cake from a surface of the filter.

3. The therapeutic device of claim 2,
wherein the one or more electrodes comprises a first electrode and a second electrode, and
wherein the filter is positioned between the first electrode and the second electrode.

4. The therapeutic device of claim 3, wherein the first electrode, the second electrode, and the filter comprise a same footprint.

5. The therapeutic device of claim 2, further comprising:
an antenna coupled to the housing and in electrical communication with the electronic circuitry, wherein the antenna is configured to wirelessly receive electromagnetic energy, and provide an electrical current to the electronic circuitry.

6. The therapeutic device of claim 1, further comprising an inlet filter coupled to the housing and positioned between the ingress of the passive pressure relief valve and the inlet of the housing, wherein a pore size of the inlet filter is at least two times larger than a pore size of the filter.

7. The therapeutic device of claim 6, wherein the pore size of the inlet filter is between 8 μm and 0.4 μm.

8. The therapeutic device of claim 7, wherein the pore size of the filter is between 100 nm and 200 nm.

9. The therapeutic device of claim 6, further comprising:
a pump configured to pump the aqueous humor toward the inlet filter.

10. The therapeutic device of claim 9, further comprising:
a pressure sensor configured to monitor a pressure of the aqueous humor; and
electronic circuitry configured to:
receive sensor data from the pressure sensor; and
provide electrical power to the pump based on the sensor data.

11. The therapeutic device of claim 9, wherein the housing further comprises a second inlet configured for receiving the aqueous humor from an interior portion of the eye and to direct the aqueous humor into the pump.

12. The therapeutic device of claim 1, wherein the passive pressure relief valve includes a spring-biased membrane configured to open to allow passage of the aqueous humor in response to the pressure at the ingress of the passive pressure relief valve exceeding an opening pressure associated with the spring-biased membrane, and wherein the threshold is associated with a spring force of the spring-biased membrane.

13. The therapeutic device of claim 12, wherein the spring-biased membrane includes a spiral-cut spring region surrounding a sealing membrane region.

14. The therapeutic device of claim 13, wherein the passive pressure relief valve further includes a first body defining the ingress and a second body defining the egress, wherein the spring-biased membrane further comprises a flange clamped between the first body and the second body.

15. The therapeutic device of claim 1, wherein the passive pressure relief valve and the filter comprise flat surfaces arranged in parallel between the inlet of the housing and the outlet of the housing, and wherein the housing defines a flat profile such that the height of the device is less than 5 mm.

16. The therapeutic device of claim 1, wherein a pressure differential between the inlet and the outlet is substantially equal to a pressure differential between a pressure of an interior portion of the eye and a pressure at the tear film or the exterior surface of the eye.

17. The therapeutic device of claim 1, wherein the passive regulation of the flow rate by the passive pressure relief valve allows for the passive pressure relief valve to entirely maintain an inlet pressure relative to a pressure at the tear film or the exterior surface of the eye.

18. The therapeutic device of claim 1, wherein the passive pressure relief valve is configured to passively regulate flow in a reverse direction.

19. The therapeutic device of claim 1, wherein the passive regulation of the flow rate by the passive pressure relief valve allows for the therapeutic device to maintain a pressure at the inlet independent of a bleb at the outlet.

20. The therapeutic device of claim 1, wherein the passive pressure relief valve further comprises a valve opening when in the open configuration, the size of the valve opening configured to change in response to the change of the pressure of the aqueous humor.
